(19) United States
(12) Patent Application Publication
(10) Pub. No.: US 2021/0196934 A1
(43) Pub. Date: Jul. 1, 2021

(54) OIL THERAPY METHOD AND DEVICE

(71) Applicant: BUTTERWORTH BUILDING, Elrington (AU)

(72) Inventor: Diane BUTTERWORTH, Elrington (AU)

(21) Appl. No.: 17/055,298

(22) PCT Filed: May 14, 2019
(86) PCT No.: PCT/AU2019/050453

§ 371 Date: Nov. 13, 2020

(30) Foreign Application Priority Data
May 14, 2018 (AU) 2018901657

(51) Int. Cl. A61M 35/00; A61K 9/70; A61K 41/00; A61K 36/47; A61K 9/00

(52) U.S. Cl. CPC A61M 35/10; A61K 9/7084; A61M 2205/36; A61K 36/47; A61K 9/0014; A61K 41/0052

(57) ABSTRACT

A oil treatment system, method and device are disclosed. An oil impregnated treatment pad is attachable to a carrier, with a heat pad attachable to the carrier, so as to deliver the treatment to a user with a heat source adjacent to the treatment pad.

### TECHNICAL FIELD

The present invention relates to methods and devices for delivering oil based external therapies, for example castor oil, essential oils or other oil based therapies.

### BACKGROUND OF THE INVENTION

Oil based therapies have been used for many years. For example, castor oil is a natural vegetable oil ingredient which has many beneficial external uses. These include, for example, relieving muscle and joint pain, relieving menstrual pain, and treating fungal infections.

For dermal applications, in general a heat source is required to activate the oil and thereby provide the necessary therapy. The oil needs to be applied to the appropriate skin area, which presents practical issues.

For a typical use of castor oil as an external therapy, a user would use a pad formed from a fabric, such as wool or cotton flannel. The pad is then soaked in castor oil, and a source of heat such as a heat pad or hot water bottle is prepared. A plastic sheet or other barrier is placed onto a bed or similar surface for the user to lie on. The sheet serves to protect the surface of the bed from being stained. A towel is then then placed into position for the user to lie on.

The user places the soaked flannel on the desired body location. Plastic film, e.g. PVC or polyethylene cling film, is them applied to hold the flannel and oil in the desired dermal location. The user lies on the prepared towel, on top of the plastic sheet, and the heat pad or hot water bottle used to warm the oil. The user then lies or sits still for 45 to 60 minutes.

It can be seen that there various practical problems with this approach. The user is limited in movement, because of the complex set up for oil application. The preparation time is considerable and may be onerous for people with limited mobility or disabilities. Further, the castor oil tends to stain clothing and furniture, and in this procedure it has to potential to create a mess before and after the treatment. There is a risk of leakage, and the associated wrap, flannel, etc. must be gathered up and disposed of after each treatment, or cleansed or stored for re-use.

Korean application KR100692501 by Moon Hyung Dae discloses a single use delivery device for absorption of a material, the device including an adhesive layer, a endothermic heating layer and an active substance absorbed in a cloth material integral with the device.

Chinese application CN205515139 by Qichun Aidu Biotechnology Co Ltd discloses a single use physical therapy device with an adhesive layer, a non-woven fabric soaked with an oil, and a heating patch, all integral with the device.

It is an object of the present invention to provide devices and methods of application for oil based therapies that are more convenient for users.

### SUMMARY OF THE INVENTION

According to a first broad aspect, the present invention uses a separate pre-packaged oil impregnated material, so that the treatment device is assembled at the point of use.

According to a second broad aspect, a reusable treatment device includes integral pads for receiving oil, and a constraint, so that the pad may be held in the desired position.

According to first aspect, the present invention provides an oil treatment system, including a first component including a heating element and an adhesive layer adapted to adhere to skin of a user, and a second component including a separately packaged oil impregnated material, the second component being adapted to be attached to the first component, so that in use the oil impregnated material is located at a selected location on the skin of the user, the adhesive layer retains the oil impregnated material at the selected location, and the heating element delivers heat to the oil impregnated material.

According to another aspect, the present invention provides a method for applying an oil treatment, including providing a first component including a heating element and an adhesive layer adapted to adhere to skin of a user, providing a second component including a separately packaged oil impregnated material, attaching the second component to the first component, placing the oil impregnated material at a selected location on the skin of the user, sealing the oil impregnated material at the selected location using the adhesive layer, and delivers heat to the oil impregnated material.

Implementations of the present invention accordingly provide a way to deliver an oil based treatment, with associated heating, in a much more convenient and less messy way for the user. This also allows for efficient use of the treatment material, and repeatable treatment methodologies to be used.

### BRIEF DESCRIPTION OF THE DRAWINGS

Illustrative implementations of the present invention will now be described with reference to the accompanying figures, in which:

FIG. 1 shows a castor oil patch system according to a first embodiment of the present invention; and

FIG. 2 shows a re-usable castor oil delivery device according to another aspect of the present invention.

### DETAILED DESCRIPTION OF THE INVENTION

The present invention will be described with reference to some specific embodiments. It will be appreciated that other implementations and variations are possible within the general scope of the invention disclosed. Moreover, aspects of the different implementations disclosed may be arranged in different combinations.

The implementations are described in the specific context of delivering castor oil to the skin of a user. However, it will be appreciated that methods and devices can be employed for other oil based treatments, for example for essential oils or other treatments. The present invention is applicable to conventional or alternative treatments.

The implementations described use a pad of flannel type material, formed from cotton or wool. It is preferred that this is formed from at least 3 layers of fabric. Other suitable woven or non-woven materials may be used. It will be understood that the pad needs to be able to be impregnated with and hold a suitable quantity of the oil for application to the skin. The area and thickness of the pad may be made suitable to the intended use and application.

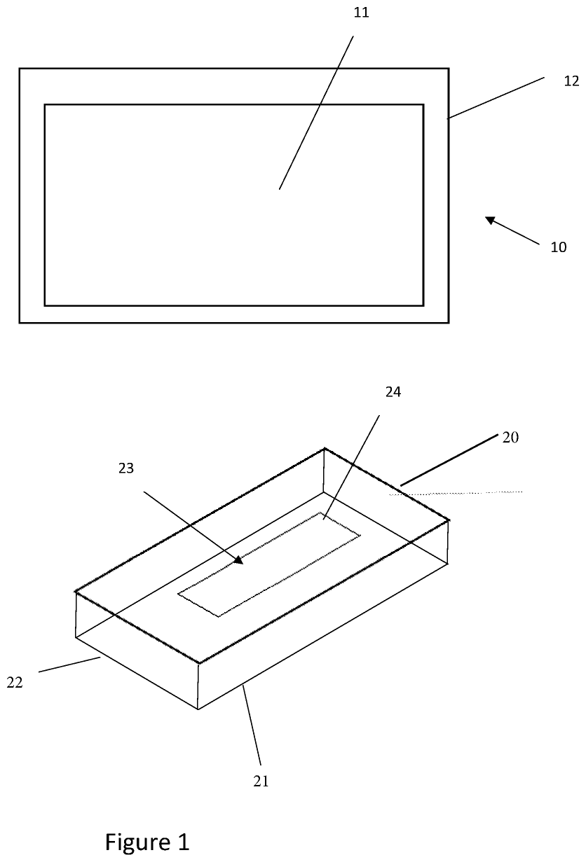

FIG. 1 is a schematic view of a device having a first component 10 and a second component 20. The first component includes a heat pack, for example an exothermic heat pack 11, and an adhesive film 12. Heat pack 12 is bonded to film 12. Film 12 has an adhesive suitable to retain film 12 in contact with the skin of a user, but also being removable when the device is no longer required. A protective, removable layer (not shown) is provided to cover the adhesive until the device is used. In some implementations, the adhesive covers the entire surface. In others, it is only present in a band around the periphery of the film.

Second component 20 is separately packaged from the first part, for example in a separately sealed bag. It provides the oil, impregnated into pad 22. The front face 21 is placed so as to engage the skin of the user at the selected location. Face 23 has an adhesive, for example provided by a double sided tape 24, in order to adhere to the film 12 in use. In other implementations, the adhesive may be provided only by film 12, and surface 23 only presents a suitable interface for the adhesive to grip.

It will be appreciated that the oil soaked material will be difficult for adhesives to grip. In one implementation, an adhesive or tape is applied to the rear of the pad before oil is soaked into the pad. In another implementation, oil is applied to the front face, and an adhesive or tape layer is applied before the oil has a chance to soak to the bottom layer. In either of these implementations, a barrier layer, for example applied as a spray, may be applied to the rear of the pad before any oil is applied. Alternative implementations using barrier layers in the pad are possible.

The oil may be applied to the material using any suitable approach. For example, the oil may be applied using a screen printing type process, and a weighted roller used to force the oil into the layers of the oil soaked material.

It will also be appreciated that the adhesive for affixing the first component to the second component must be of a type that, once affixed, later oil contact will not substantially weaken or dissolve the bond. As an alternative to adhesives, alternative attachments such as a hook and loop system (e.g. Velcro®), magnetic attachments, or other means may be used.

Keeping the oil impregnated section in a separate sealed enclosure greatly reduces the chance of spillage and staining from the package. It also allows for the condition of the oil to the optimised, as it is not in contact with the other materials of the treatment system.

The device can be readily removed and disposed of as a whole after the required treatment. It is also easy to apply several devices at once if that is indicated, for example for treating separate skin areas.

It will be appreciated that the first component may be packaged in a similar mode to a conventional dressing or heating pack. The second component should be packaged in a sealed enclosure, for example formed from a suitable plastic material, to retain the oil and minimise contact with the atmosphere until intended for use.

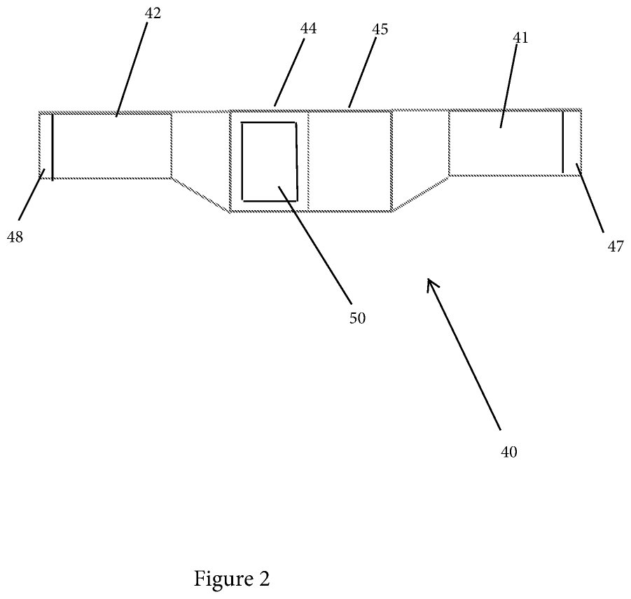

FIG. 2 illustrates a second implementation of the present invention. In this implementation, the device 40 is reusable, rather than single use. Belt 40 includes two pocket sections 44, 45, formed from a fabric, for example an open weave fabric or any other suitable material. Belt sections 41, 42 are formed from an extensible material. In the illustrative carrier, a fastening system, for example a hook and loop (e.g. Velcro®) engagement can be provided at the respective ends 47, 48 to allow the carrier to be easily fastened. It could alternatively have a buckle, press studs, pins and holes, or any other connection, ideally to allow for a degree of adjustment. In other implementations, the carrier material is simply stretched around the body and then released.

Pocket sections 44, 45 are intended to receive respective heat pads (not shown). An oil soaked pad 50 can be affixed to either or both of the pocket sections. The pad 50 may be formed in a similar manner to that described above, using layers of flannel or other fabric, and providing a mechanism for affixing the pad to the pocket 44, 45. This may be a more or less permanent attachment, using an adhesive double sided tape or similar material, or may be removable, for example using a hook and loop type attachment.

The pad 50 is mounted on a belt or other carrier 40. As will be explained below, the belt is wrapped around and fastened so that pad 50 is held in the selected position on the user.

It will be understood that the exact dimensions of belt 40 and pad 50 may vary depending upon an intended area of use—for example, a much longer belt will be required for the torso than to go around a limb. The carrier may be, for example, a stretchable material, a shaped garment, a sleeve, or any other shape or form suitable to hold the pad in position.

This implementation may be sold with a pad and a separate oil dispenser, for example a syringe, to soak the pad. Alternatively, it could be sold with a pre-soaked pad, and a resealable cover. In another form, the pad could be removable and stored separately, and optional replacement pre-impregnated pads used. In the illustrated example, the device 40 may be folded up, with the pads 50 facing each other (if two are used), and the whole device stored in, for example, as plastic container to prevent any undesired staining.

The heat pad is received in a pouch or similar attachment 44, 45, which sits on the outside of the carrier 40, adjacent to the pad 50.

In both implementations, the heat pad is preferably of the exothermic type, supplied sealed from air, and which heats up in a controlled way due to a reaction with oxygen. Any suitable commercially available device may be used. Alternative heat pads and device could be used if desired in an alternative implementation.

Devices according to the implementations described can be made into different sized devices, for example, large patches such as 30 cm×30 cm, and can also be made into specific shapes for specific uses, such as tubular for arms, knees, and elbows. The invention can also include reusable knee and elbow guards with the castor oil patch. Further, a reusable bandage can be provided with the patch located inside.

It is to be understood that, if any prior art information is referred to herein, such reference does not constitute an admission that the information forms part of the common general knowledge in the art, in Australia or any other country.

1. An oil treatment system, including a first component including a heating element and an adhesive layer adapted to adhere to skin of a user, and a second component including a separately packaged oil impregnated material, the second component being adapted to be attached to the first component, so that in use the oil impregnated material is located at a selected location on the skin of the user, the adhesive layer retains the oil impregnated material at the selected location, and the heating element delivers heat to the oil impregnated material.

2. An oil treatment system according to claim 1, wherein the oil impregnated material includes an adhesive attachment for attaching the oil impregnated material to the first component.

3. An oil treatment system according to claim 1, wherein the oil impregnated material is formed from multiple layers, and the adhesive attachment is affixed to the back layer.

4. An oil treatment system according to claim 1, wherein the adhesive layer operatively seals the oil impregnated material to prevent leakage.

5. An oil treatment kit, including a first component and a second component according to claim 1.

6. A method for applying an oil treatment, including providing a first component including a heating element and an adhesive layer adapted to adhere to skin of a user, providing a second component including a separately packaged oil impregnated material, attaching the second component to the first component, placing the oil impregnated material at a selected location on the skin of the user, sealing the oil impregnated material at the selected location using the adhesive layer, and delivering heat to the oil impregnated material.

7. An oil treatment device, including a carrier, a separately packaged treatment pad formed from oil impregnated material, the treatment pad being adapted to be attached by a user to the carrier, the carrier being adapted to be retained on the body of a user, the device further including an attachment for receiving a heat pad, so that the treatment pad is operatively positioned facing the skin of a user, and the heat pad when fitted is outside the carrier so as to provide heat to the treatment pad.

8. An oil treatment device according to claim 7, wherein the carrier is reusable.

9. An oil treatment device according to claim 8, wherein the treatment pad is supplied separately with an oil treatment pre-loaded, and the pad is attached by the user to the carrier.

10. An oil treatment device according to claim 8, wherein the heat pad is retained in a pocket.
